(19) United States
(12) Patent Application Publication
(10) Pub. No.: US 2010/0317709 A1
(43) Pub. Date: Dec. 16, 2010

(54) SPHINGOSINE-1-PHOSPHATE (S1P) RECEPTOR COMPOUNDS

(75) Inventors: Johannes Brussee, Rijinsburg (NL); Adriaan Pieter Ijzerman, Haarlem (NL); Astrid Eline Alewijnse, Amsterdam (NL); Stephan Leonard Maria Peters, Amsterdam (NL); Margot Wilhelmia Beukers, Leiden (NL)

Correspondence Address: BROWDY AND NEIMARK, P.L.L.C.;624 NINTH STREET, NW, SUITE 300, WASHINGTON, DC, 20001-5303, US

(73) Assignee: UNIVERSITEIT LEIDEN

(21) Appl. No.: 12/742,988

(22) PCT Filed: Nov. 13, 2008
(86) PCT No.: PCT/IB08/03056

§ 371 Date: Aug. 23, 2010

(30) Foreign Application Priority Data
Nov. 14, 2007 (GB) GB0722340.7

(51) Int. Cl. A61K 31/381; C07D 333/38; C07D 333/68; C07D 409/02; A61P 9/00; A61P 11/00; A61P 35/00

(52) U.S. Cl. 514/414; 549/68; 514/447; 549/57; 514/443; 548/468

(57) ABSTRACT

The invention relates to compounds, in particular 2-amino-3,4,5,-trisubstituted thiophenes, pharmaceutical compositions containing them and the uses of said compounds and compositions for diseases related to sphingosine-1-phosphate (S1P) receptors, predominantly S1P3 receptors. The diseases include cardiovascular diseases, atherosclerosis, cancer, pulmonary oedema, autoimmune disorders and Adult Respiratory Distress Syndrome.

### FIELD OF INVENTION

The present invention relates to a particular novel category of 2-amino-3,4,5,-trisubstituted thiophenes, pharmaceutical compositions containing them and their uses of said compounds and compositions for diseases related to sphingosine-1-phosphate (S1P) receptors, predominantly S1P₃ receptors.

### BACKGROUND OF THE INVENTION

Several years before the de-orphanization of the Endothelial differentiation gene (EDG) receptors the effects of sphingosine-1-phosphate (S1P) on endothelial cells were already recognized although the mechanism/targets were unknown. Originally, S1P was thought to act as an intracellular messenger but there is still no conclusive evidence for such an intracellular target. Identification and characterization of the family of genes involved in S1P signaling in 1998 was thus an important step in this field of research. Five members of the S1P receptor family, originally named EDG receptors, have been identified so far named S1P₁, through S1P₅ (Chun et al., 2002) (1). Being part of the G-protein coupled receptor family, S1P receptors signal through heterotrimeric G-proteins. Because each S1P receptor subtype activates a different set of G-proteins the final cellular effect of activation of each receptor subtype will be different (see for extensive review on signaling of S1P receptors, Sanchez and Hla, 2004) (2). The S1P₁, S1P₂ and S1P₃ receptor are ubiquitously expressed and are the most important receptors in the cardiovascular system. The S1P₄ and S1P₅ receptor show a more restricted expression pattern and the first is predominantly expressed in the lung and lymphoid system whereas the S1P₅ receptor is mainly expressed in brain tissue.

S1P receptors are involved in many biological processes and may as such have an important role in many pathophysiological disease states such as atherosclerosis, cancer and auto-immunity. Despite their importance the number of ligands that specifically interact with S1P receptors is very limited. In addition, subtype specific S1P ligands are as good as non-existing. The absence of (subtype) selective S1P ligands generally hampers pharmacological research of these receptors.

### S1P₃ Receptors

The S1P₃ receptor subtype plays a critical role in cardiac rhythm and lung epithelial barrier function. The role of the S1P₃ receptor in cardiac rhythm was recognized due to the cardiac side-effects of FTY720, a non-specific S1P agonist which is momentarily in clinical trials for the treatment of MS. Studies using S1P₃ knock-out mice, subsequently, suggested that the negative chronotropic effect of FTY720 was predominantly related to the S1P₃ receptor expressed in atrial myocytes but also on the sino-atrial node (Forrest et al., 2004, (3); Sanna et al., 2004) (4). In addition, it has been shown recently that the cardioprotective effects of high density lipoproteins are mediated via S1P₃ receptors (Theilmeier, 2006) (5). Furthermore, also the anti-atheroslerotic action of FTY720 in ApoE−/− mice is believed to be mediated for a major part by S1P₃ receptors (Keul et al., 2007) (6). Consequently, S1P₃ agonists may be useful as cardioprotective compounds.

In lung, S1P₃ is exclusively expressed in pulmonary epithelium and activation of S1P₃ results in degradation of ZO-1 and claudin leading to a decreased epithelial tight junction formation. This will finally lead to the development of paracellular gaps and pulmonary edema. As such, selective S1P₃ agonists and/or antagonists would be of help to elucidate the mechanisms involved in Adult Respiratory Distress Syndrome and could eventually also be of therapeutic importance in this disease.

### SUMMARY OF THE INVENTION

Considering the above-described circumstances, the present inventors made intensive study. As a result, they found surprisingly a particular category of 2-amino-3,4,5,-trisubstituted thiophenes that can be used very attractively to treat, S1P receptor mediated, predominantly S1P₃ receptor mediated conditions.

According to a first aspect the present invention provides the use of a compound of the general formula (1):

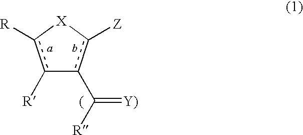

or a salt thereof,
wherein
X represents oxygen sulphur, NH or C₁-C₄ alkyl substituted NH;
C═Y is optional but when present, Y represents oxygen, sulphur, NH, or C₁-C₄ alkyl substituted NH:
Z represents hydrogen, halogen, amide, hydroxyl, substituted or unsubstituted alkyl, substituted or unsubstituted alkenyl, substituted or unsubstituted aryl or heteroaryl, substituted or unsubstituted aralkyl, alkoxyl, amino, mono- or di-C₁-C₄ alkyl substituted amino, sulphydryl, carbonyl, carboxyl, acrylate, methacrylate, vinyl, styryl, sulphonate, sulphonic acid, or quaternary ammonium;
R represents hydrogen, halogen, substituted or unsubstituted alkyl, substituted or unsubstituted alkenyl, substituted or unsubstituted alkynyl, or substituted or unsubstituted —(CH₂)ₙ-aryl;
R′ represents hydrogen, halogen, substituted or unsubstituted alkyl, substituted or unsubstituted alkenyl, substituted or unsubstituted alkynyl, or substituted or unsubstituted —(CH₂)ₙ-aryl; or R,R′ are linked together to form a ring which may be saturated or unsaturated, substituted or unsubstituted and has three to seven members;
R″ represents hydrogen, —(CH₂)ₙ-hydroxyl, halogen, acyl, thio-acyl, seleno-acyl, (substituted) alkyl, (substituted) alkenyl, (substituted) alkynyl, or (substituted) —(CH₂)ₙ-aryl;
bonds a and b, shown in dashed line, may be present or absent;
and n is a number in the range of from 0 to 10;
in the preparation of a medicament for the treatment or prophylaxis of a condition mediated by a sphingosine 1 phosphate (S1P) receptor of a subject.

Preferably where Z is substituted or unsubstituted alkyl, alkoxy, aralkyl or alkenyl, the alkyl or alkenyl group is C₁-C₄. Preferably Z is NH₂. Preferably X is sulphur. Preferably Y is oxygen.

There is also provided use of the aforementioned compounds of formula (1) in the treatment or prophylaxis of a condition mediated by an S1P receptor of a subject.

It will be understood that compounds of formula (1) may be chiral and as such all enantiomeric forms are encompassed by the invention. The medicament may include racemic mixtures or individual enantiomers of a selected compound.

Compounds of formula (1) may be selective or specific for different S1P receptors. Certain preferred examples as described hereafter are specific for S1P₃ receptors. The selectivity and/or specificity of the compounds of the invention increases their potential utility in use against conditions that are mediated by the S1P receptors. It will be understood that conditions amenable to treatment by the compounds of the invention may be directly affected by the activity of the S1P receptors or may be those where the activity at the S1P receptors is part of a biochemical pathway that is relevant to the condition being treated and as such action on a S1P receptor has a resulting effect downstream.

The compounds of the present invention modulate the activity of S1P receptors. Preferably, the compounds modulate the activity of S1P₃ receptors. The modulation of the activity of the receptors caused by the compounds of formula (1) can be, for example, by acting as agonists or as antagonists at the receptor. Thus diseases such as amongst others cardiovascular disorders, can very attractively be treated and/or prevented. The conditions that can be treated by compounds of formula (1) include atherosclerosis, cancer, pulmonary oedema, autoimmune disorders and Adult Respiratory Distress Syndrome.

More preferably the compound of formula (1) has the structure of formula (2):

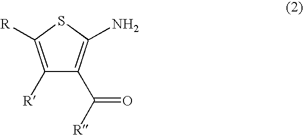

wherein R,R′ and R″ have the same meaning as before.

A number of compounds of formula (2) are known as allosteric modulators for the Adenosine A1 receptor (Refs 7 and 9 to 14) but their activity in respect of S1P receptors and hence their utility in conditions mediated by these receptors has hitherto been unknown. In some cases the compounds of formula 1 may modulate activity at both the Adenosine A1 receptor and one or more S1P receptors resulting in enhanced improvement of a condition that is mediated by both adenosine and S1P receptors.

Where RR′ forms a ring, in compounds of Formula (2), preferably RR′ is

—(CH₂)₄— i.e. the compounds have a six membered ring fused with the thiophene ring and have the general formula (3):

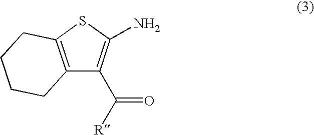

wherein R″ has the same meaning as before.

Preferably R″ is phenyl or substituted phenyl. Most preferably, the compound of general formula (3) is selected from the group consisting of:
(2-Amino-4,5,6,7-tetrahydrobenzo[b]thiophen-3-yl) (4-chlorophenyl)methanone (LUF 5480);
(2-Amino-4,5,6,7-tetrahydrobenzo[b]thiophen-3-yl) (4-bromophenyl)methanone (LUF5490);
Methyl 4[(2-Amino-4,5,6,7-tetrahydrobenzo[b]thiophen-3-yl)-carbonyl]benzoate (LUF5492);
(2-Amino-4,5,6,7-tetrahydrobenzo[b]thiophen-3-yl) (2-chlorophenyl)methanone (LUF5532);
(2-Amino-4,5,6,7-tetrahydrobenzo[b]thiophen-3-yl) (3-chlorophenyl)methanone (LUF5473);
(2-Amino-4,5,6,7-tetrahydrobenzo[b]thiophen-3-yl) (3-iodophenyl)methanone (LUF5533);
(2-Amino-4,5,6,7-tetrahydrobenzo[b]thiophen-3-yl) (4-methylphenyl)methanone, (LUF5527); and
(2-Amino-4,5,6,7-tetrahydrobenzo[b]thiophen-3-yl)[3-(trifluoromethyl)phenyl]methanone (LUF5469), or a salt thereof.

According to a second aspect the present invention provides a method of treatment of an S1P receptor mediated condition comprising the administration of an effective amount of a compound of formula (1) to a subject. The condition is preferably an S1P₃ receptor mediated condition.

According to a third aspect the present invention provides a compound of the general formula (I):

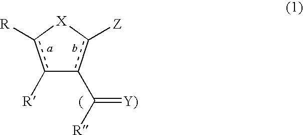

or a salt thereof,
wherein
X represents oxygen sulphur, NH or C₁-C₄ alkyl substituted NH;
C═Y is optional but when present, Y represents oxygen, sulphur, NH, or C₁-C₄ alkyl substituted NH:
Z represents hydrogen, halogen, amide, hydroxyl, substituted or unsubstituted alkyl, substituted or unsubstituted alkenyl, substituted or unsubstituted aryl or heteroaryl, substituted or unsubstituted aralkyl, alkoxyl, amino, mono- or di-C₁-C₄ alkyl substituted amino, sulphydryl, carbonyl, carboxyl, acrylate, methacrylate, vinyl, styryl, sulphonate, sulphonic acid, or quaternary ammonium;
R represents hydrogen, halogen, substituted or unsubstituted alkyl, substituted or unsubstituted alkenyl, substituted or unsubstituted alkynyl, or substituted or unsubstituted —(CH₂)ₙ-aryl;
R′ represents hydrogen, halogen, substituted or unsubstituted alkyl, substituted or unsubstituted alkenyl, substituted or unsubstituted alkynyl, or substituted or unsubstituted —(CH₂)ₙ-aryl; or R,R′ are linked together to form a ring which may be saturated or unsaturated, substituted or unsubstituted and has three to seven members;
R″ represents hydrogen, —(CH₂)ₙ-hydroxyl, halogen, acyl, thio-acyl, seleno-acyl, (substituted) alkyl, (substituted) alkenyl, (substituted) alkynyl, or (substituted) —(CH₂)ₙ-aryl;
bonds a and b, shown in dashed line, may be present or absent;
and n is a number in the range of from 0 to 10.

In the context of the present invention the term ‘a condition mediated by a sphingosine 1-phosphate receptor (S1P)” is intended to include disease states or conditions characterised by their responsiveness to treatment with a sphingosine 1-phosphate receptor modulating compound, e.g. a compound og general formula (1), where the treatment causes a significant diminishment of at least one symptom or effect of the condition.

In the context of the present invention by the term ‘alkyl’ it is meant any saturated hydrocarbon, either branched or unbranched comprising from 1 to about 30 carbon atoms. This includes straight-chained alkyl groups, branched-chained alkyl groups, cycloalkyl(alicyclic) groups, alkyl-substituted cycloalkyl groups, and cycloalkyl-substituted alkyl groups. This term further includes alkyl groups, which can further include oxygen, nitrogen, sulphur or phosphorous atoms replacing one or more carbons of the hydrocarbon backbone. In preferred embodiments, a straight or branched chain has 30 or less carbon atoms in its backbone, and more preferably 20 carbon atoms or less. Likewise, preferred cycloalkyls have from 3-10 carbons, and more preferably 3-7 carbons in the ring-structure.

In the context of the present invention the term

‘—(CH₂)ₙ-hydroxyl’ means a short straight alkyl chain between the hydroxyl group and the drawn structure, where n can range of from 0 up to and including 10.

In the context of the present invention the terms ‘acyl’, ‘thio-acyl’ and ‘seleno-acyl’ refer to compounds of the kind ‘C(O)A, ‘C(S)A’, and ‘C(Se)A’, respectively, where A in turn represents hydrogen, (substituted) alkyl, (substituted) alkenyl, (substituted) alkynyl, or (substituted) —(CH₂)ₙ-aryl.

In the context of the present invention the term

‘—(CH₂)ₙ-aryl’ means a short straight alkyl chain between the (substituted) aryl group and the drawn structure, where n can range of from 0 up to and including 10.

In the context of the present invention the term ‘aryl’ as used herein, refers to aromatic groups which can include 5- and 6-membered single-ring groups, with 0 to 4 heteroatoms, for example benzene, pyrrole, furan, thiophene, imidazole, triazole, tetrazole, pyrazole, pyridine, pyrazine, pyridazine and pyrimidine, and the like. The aryl groups can suitably include polycyclic fused aromatic groups such as naphthyl, quinolyl, indolyl, benzoxazole, benzothiazole and the like. Those aryl groups containing heteroatoms may also be referred to as heteroaryls or heteroaromatics. The aromatic ring may be substituted at one or more ring positions, with such substituents as described herein. Aryl groups can also be fused or bridged with alicyclic or heteroalicyclic rings which are not aromatic.

In the context of the present invention the term ‘substituted’ is intended to include substituents replacing hydrogen on one or more of the carbons of a moiety. Such substituents suitably include, for example, halogen, hydroxyl, alkylcarbonyloxy, arylcarbonyloxy, alkoxycarbonyloxy, aryloxycarbonyloxy, carboxylate, alkylcarbonyl, alkyloxycarbonyl, aminocarbonyl, alkylthiocarbonyl, alkyoxyl, phosphate, phosphonate, phosphinato, cyano, amino (including alkylamino, dialkylamino, arylamino, diarylamino, alkylarylamino), acylamino, (including alkylcarbonylamino, arylcarbonylamino, carbamyl and ureido), amidino, imino, sulfhydryl, alkylthio, arylthio, thiocarboxylate, sulfates, sulfonato, sulfamoyl, sulfonamido, nitro, trifluoromethyl, azido, heterocyclyl, alkylaryl, or an aromatic or heteroaromatic moiety. It will be understood to those skilled in the art that the moieties substituted on the (unsaturated and saturated) carbon chain can themselves be substituted, if appropriate. The term substituted also

includes the replacement of one or more of the carbon atoms in a moiety with a heteroatom.

In the context of the present invention the term ‘heteroatom’ refers to an atom of any element other than carbon or hydrogen. Preferred heteroatoms are oxygen, nitrogen, sulphur and phosphorus.

In the context of the present invention the terms ‘alkenyl’ and ‘alkynyl’ refer to unsaturated aliphatic groups analogous in length and possible substitution to the alkyls described above, but that contain at least one double or triple bond, respectively.

In the context of the present invention the term ‘halogen’ refers to an atom of group VII of the periodic table. Preferred halogens are fluorine, chlorine, bromine, iodine.

In the context of the present invention salts of the compound of the present invention are meant to include any physiologically acceptable salt. The term ‘physiologically acceptable salt’ refers to any non-toxic alkali metal, alkaline earth metal, and ammonium salts commonly used in the pharmaceutical industry, including the sodium, potassium, lithium, calcium, magnesium, barium ammonium and protamine zinc salts, which can be prepared by methods known in the art. The term also includes non-toxic acid addition salts, which are generally prepared by reacting the compounds of the present invention with a suitable organic or inorganic acid. The acid addition salts are those which retain the biological effectiveness and properties of the free bases and which are not biologically or otherwise undesirable. Examples include those derived from mineral acids, and include, inter alia, hydrochloride, hydrobromic, sulphuric, nitric phosphoric, metaphosphoric and the like. Organic acids include, inter alia, tartaric, acetic, proprionic, citric, malic, malonic, lactic, fumaric, benzoic, cinnamic, mandelic, glycolic, gluconic, pyruvic, succinic, salicylic and arylsulfonic, e.g. p-toluenesulfonic, acids.

According to a fourth aspect, the present invention provides a pharmaceutical composition comprising as active ingredient one or more compounds of the general formula (1):

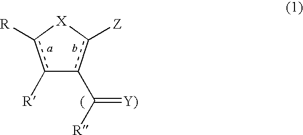

or a salt of said compound(s), wherein R, R′, R″, X, Y, Z, a and b have the meaning as defined herein before.

The present invention also relates to a pharmaceutical composition comprising as active ingredient one or more compounds according to the present invention. The compound according to the present invention can be used as such. However, also a salt or a solvate of the compound may be used. It will be understood that such salt or solvate should be pharmaceutically acceptable. The skilled person will further understand that the pharmaceutical composition will also comprise a suitable pharmaceutical carrier.

As will be detailed in Table 1, the compounds of the present invention are biologically active.

The term ‘biologically active’ indicates that the compound of the present invention has some sort of a biological activity, for example, a measurable effect, a modulation, on a target receptor. As will be detailed hereinafter, the exemplary compounds of formula (1) activate S1P₃ receptors, thus acting as sphingosine 1-phosphate receptor agonists. In another embodiment, the term includes antagonistic effects, e.g. diminishment of the activity or production of mediators which result from the (over)-stimulation of sphingosine 1-phosphate receptor(s).

The terms ‘modulate’, ‘modulation’, and ‘modulation’ used herein refer to the effect of increasing decreasing or otherwise changing the activity of a receptor.

The term agonist’ used herein refers to a molecule that binds to a receptor and activates the receptor. The term antagonist refers to a molecule that binds to a receptor and causes diminishment of the activity.

In the pharmaceutical composition according to the present invention the active ingredient is present in an effective amount. The term ‘effective amount’ for the purposes described herein is that determined by such considerations as are known to those versed in the art. The amount must be sufficient to achieve a desired therapeutic effect, e.g. to treat a disease or disorder.

The terms ‘treat’, ‘treating’ and ‘treatment’ refer to the administering of a therapeutic amount of the compound or pharmaceutical composition of the present invention which is effective to ameliorate undesired symptoms associated with a disease, to prevent the manifestation of such symptoms before they occur, to slow down the progression of a disease, to slow down the deterioration of symptoms, to slow down the irreversible damage caused by the chronic stage of a disease, to lessen the severity of, or cure a disease, to improve survival rate or more rapid recovery, to prevent the disease from occurring, or a combination of two or more of the above.

The disease is preferably associated with the biological action of S1P₃ receptors wherein the compound of the present invention acts as a sphingosine 1-phosphate receptor agonist. For example agonists of S1P₃ receptors have been implicated as compounds that may be used in the treatment of cardiovascular diseases e.g. atherosclerosis.

The pharmaceutical composition of the present invention may further comprise pharmaceutically acceptable additives.

Further, the term ‘pharmaceutically acceptable additives’ used herein refers to any substance combined with said compound and include, without being limited thereto, diluents, excipients, carriers, solid or liquid fillers or encapsulating materials which are typically added to formulations to give them a form or consistency when it is given in a specific form, e.g. in tablet form, as a simple syrup, aromatic powder, and other various elixirs. The additives may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavour, etc.

Preferably, the additives are inert, non-toxic materials, which do not react with the active ingredient of the invention. Yet, the additives may be designed to enhance the binding of the agent to its receptor. Further, the term additive may also include adjuvants, which, by definition, are substances affecting the action of the active ingredient in a predictable way. The additive can be any of those conventionally used and are only limited by chemico-physical considerations, such as solubility and lack of reactivity with the compound of the invention, and by route of administration. The active agent of the invention may be administered orally to the patient. Conventional methods such as administering the compound/s in tablets, suspensions, emulsions, capsules, powders, syrups and the like are usable. For oral administration, the composition of the invention may contain additives for facilitating oral delivery of the compound/s of the invention. Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatine type containing, for example surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatine, guar gum, colloidal silicon dioxide, croscarmellose sodium talc, magnesium stearate, calcium stearate, zinc stearate, stearic acid, and other excipients, colorants, diluents, buffering agents, and pharmacologically compatible carriers. Lozenge forms can comprise the active agent in a flavour, usually sucrose and acacia or tragacanth, as well as pastilles comprising the active ingredient in an inert base, such as gelatine and glycerine, or sucrose and acacia, emulsions, gels, and the like. Such additives are as such known in the art.

Alternatively, the compound/s may be administered to the patient parenterally. In this case, the composition will generally be formulated in a unit dosage injectable form (solution, suspension, emulsion). Pharmaceutical formulation suitable for injection may include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, lipid polyethylene glycol and the like), suitable mixtures thereof and vegetable oils.

Proper fluidity can be maintained, for example, by the use of a coating such as lecithin, by the maintenance of the required particle size in the case of dispersion and by the use of surfactants. Non-aqueous vehicles such as cottonseed oil, sesame oil, olive oil, soybean oil, corn oil, sunflower oil, or peanut oil and ester, such as isopropyl myristate, may also be used as solvent systems for the composition of the present invention.

Suitable fatty acids for the use in parenteral formulations include oleic acid, stearic acid, and isostearic acid. Ethyl oleate and isopropyl myristate are examples of suitable fatty acid esters.

Suitable detergents for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefinic sulfonates, alkyl, olefin, ether and monoglyceride sulfates, and sulfosuccinates, (c) non-ionic detergents such as, for example, fatty amine oxides, fatty acid alkanolamides, and polyoxy-ethylenepolypropylene copolymers, (d) amphoteric detergents such as, for example, alkyl-S-aminopropionates, and 2-alkyl-imidazoline quarternary ammonium salts, and mixtures thereof.

Further, in order to minimise or eliminate irritation at the site of injection, the compositions may contain one or more non-ionic surfactants having a hydrophile-lipophile balance (HLB) from about 12 to about 17. Suitable surfactants include polyethylenesorbitan fatty acid esters, such as sorbitan monooleate and the high molecular weight adducts of ethylene oxide with a hydrophobic base, formed by the condensation of propylene oxide with propylene glycol.

The choice of an additive will be determined in part by the particular compound of the present invention, as well as by the particular method used to administer the composition.

Notwithstanding the above, the composition of the present invention may include one or more of the compounds of the present invention and may compromise other biologically active substances, to provide a combined therapeutic effect.

The compounds and compositions of the present invention as set forth hereinabove and below are administered and dosed in accordance with good medical practice, taking into account the clinical conditions of the individual patient, the site and method of administration, scheduling of administration, individual's age, sex, body weight and other factors known to medical practitioners.

The dose may be single doses or multiple doses over a period of several days. The treatment generally has a length proportional to the length of the disease process and drug effectiveness and the individual species being treated. Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments, until the optimum effect under the circumstances is reached. Exemplary dosages range from about 0.01 mg/kg body weight to about 10 mg/kg body weight of the subject being treated per day.

The invention has been described in an illustrative manner, and it is to be understood that the terminology which has been used, is intended to be in the nature of words of description rather than of limitation. Obviously, many modifications and variations of the present invention are possible in light of the above teaching. It is therefore, to be understood that within the scope of the appended claims, the invention may be practised otherwise than as specifically described hereinafter.

Throughout this application various publications are referred to by a number. Full citations for the publications are listed hereinafter. The disclosure of these publications in their entireties is hereby incorporated by reference into the application in order to more fully describe the state of the art to which this invention pertains.

### DETAILED DESCRIPTION

### Example of Synthetic Methods

The compounds of the present invention (Formula 1) may be prepared by several synthetic procedures. For example, the synthetic route to obtain some 2-amino-3,4,5,-trisubstituted thiophene derivatives (Formula 2) is depicted in the scheme herein below:

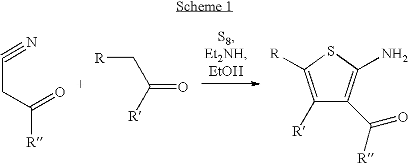

According to this scheme, the appropiate carbonyl compounds were reacted with benzoylacetonitrile derivatives and sulfur in ethanol in the presence of diethylamine to provide the products. These benzoylacetonitrile derivatives, when not commercially available, were synthesized by condensation of the acetonitrile anion with the appropiate substituted methyl benzoates.

### Specific Examples

### 2-Amino-3,4,5,-trisubstituted thiophenes

This invention is further described in the following specific examples, which do not limit the scope of the invention described in the claims.

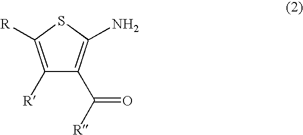

The examples detailed here of the general formula (2) are synthesised according to the route detailed in Scheme 1.

### Chemistry—General

Chemicals and Solvents All reagents were obtained from commercial sources and all solvents were of an analytical grade.

Chromatography Thin-layer chromatography (TLC) was carried out using Merck silica gel plastic backed F₂₅₄ plates, visualised under UV (254 nm).

Instruments and Analysis Elemental analyses were performed for C,H,N (Leiden Institute of Chemistry, Leiden University, The Netherlands). ¹H and ¹³C NMR spectra were recorded on a Bruker AC 200 (¹H NMR, 200 MHz; ¹³C NMR, 50.29 MHz) spectrometer with tetramethylsilane (TMS) as an internal standard. Chemical shifts are reported in ppm (δ) relative to this. Melting points were determined on a Büchi melting point apparatus and are uncorrected. Mass Spectra were measured on a Finnigan MAT TSQ-70 spectrometer equipped with an electrospray interface for ESI experiments. Spectra were collected by constant infusion of the analyte dissolved in methanol. ESI is a soft ionisation technique resulting in protonated, sodiated species in positive ionisation mode and deprotonated species in the negative ionisation mode.

### Synthetic Procedures

### (Klein, J M C, 1999, 42, 3629-3635) (7)

General procedure for the synthesis of 2-amino-3-benzoylthiophenes. To a suspension of 5 mmol of carbonyl compound, 5 mmol of benzoylacetonitrile derivative, and 5.05 mmol of sulfur in 1.5 mL of ethanol was added 1 mL of diethylamine. The mixture was stirred for 2 h at 50° C. The mixture was cooled to room temperature. If the product crystallized from the crude reaction mixture, the precipitate was collected and recrystallized from the appropiate solvent. When no crystallization occurred, the mixture was evaporated, purified by column chromatography and crystallized from the appropiate solvent.

### LUF5463

(2-Amino-4,5-dimethyl-3-thienyl)(phenyl)methanone was prepared as described above, starting from methyl ethyl ketone and benzoylacetonitrile: mp 133-134° C., ¹H NMR (CDCl₃) δ 1.54 (s, 3H CH₃), 2.13 (s, 3H, CH₃), 6.40 (bs, 2H, NH₂), 7.39-7.54 (m, 5H, Hₐᵣₒₘ).

### LUF5471

(2-Amino-4,5-dimethyl-3-thienyl)(3-chlorophenyl)methanone was prepared as described above, starting from methyl ethyl ketone and 3-chlorobenzoylacetonitrile: mp 114-116° C., ¹H NMR (CDCl₃) δ 1.52 (s, 3H CH₃), 2.10 (s, 3H, CH₃), 6.75 (bs, 2H, NH₂), 7.30-7.49 (m, 4H, Hₐᵣₒₘ).

### LUF5487

(2-Amino-4,5-dimethyl-3-thienyl)(4-chlorophenyl)methanone was prepared as described above, starting from methyl ethyl ketone and 4-chlorobenzoylacetonitrile: mp 123-125° C., ¹H NMR (CDCl₃) δ 1.55 (s, 3H CH₃), 2.13 (s, 3H, CH₃), 7.35-7.49 (m, 6H, NH₂, Hₐᵣₒₘ).

### LUF5464

(2-Amino-4-ethyl-5-methyl-3-thienyl)(phenyl)methanone was prepared as described above, starting from 3-pentanone and benzoylacetonitrile: mp 129-131° C., ¹H NMR (CDCl₃) δ 0.71 (t, 3H CH₃), 2.09 (q, 2H, CH₂), 2.16 (s, 3H, CH₃), 6.10 (bs, 2H, NH₂), 7.30-7.65 (m, 5H, Hₐᵣₒₘ).

### LUF5468

(2-Amino-4-ethyl-5-methyl-3-thienyl)[3-(trifluoromethyl)phenyl]methanone was prepared as described above, starting from 3-pentanone and 3-trifluoromethylbenzoylacetonitrile: mp 100-101° C., ¹H NMR (CDCl₃) δ 0.68 (t, 3H CH₃), 2.00 (q, 2H, CH₂), 2.16 (s, 3H, CH₃), 6.55 (bs, 2H, NH₂), 7.54-7.80 (m, 4H, Hₐᵣₒₘ).

### LUF5472

(2-Amino-4-ethyl-5-methyl-3-thienyl)(3-chlorophenyl)methanone was prepared as described above, starting from 3-pentanone and 3-chlorobenzoylacetonitrile: mp 99-102° C., ¹H NMR (CDCl₃) δ 0.71 (s, 3H CH₃), 2.08 (q, 2H, CH₂), 2.13 (s, 3H, CH₃), 6.40 (bs, 2H, NH₂), 7.30-7.60 (m, 4H, Hₐᵣₒₘ).

### LUF5482

(2-Amino-4-ethyl-5-methyl-3-thienyl)(4-chlorophenyl)methanone was prepared as described above, starting from 3-pentanone and 4-chlorobenzoylacetonitrile: mp 108-110° C., ¹H NMR (CDCl₃) δ 0.71 (s, 3H CH₃), 2.09 (q, 2H, CH₂), 2.16 (s, 3H, CH₃), 6.00 (bs, 2H, NH₂), 7.36-7.55 (m, 4H, Hₐᵣₒₘ).

### LUF5465

(2-Amino-4,5,6,7-tetrahydrobenzo[b]thiophen-3-yl)(phenyl)methanone was prepared as described above, starting from cyclohexanone and benzoylacetonitrile: 152-153° C., ¹H NMR (CDCl₃) δ 1.40-1.62 (m, 2H CH₂), 1.68-1.80 (m, 4H, 2×CH₂), 2.48-2.55 (m, 2H, CH₂), 6.65 (bs, 2H, NH₂), 7.38-7.60 (m, 5H, Hₐᵣₒₘ).

### LUF5532

(2-Amino-4,5,6,7-tetrahydrobenzo[b]thiophen-3-yl)(2-chlorophenyl)methanone was prepared as described above, starting from cyclohexanone and 2-chlorobenzoylacetonitrile: mp 145-147° C., ¹H NMR (CDCl₃) δ 1.44-1.71 (m, 6H, 3×CH₂), 2.44-2.49 (m, 2H, CH₂), 7.21-7.37 (m, 4H, Hₐᵣₒₘ).

### LUF5469

(2-Amino-4,5,6,7-tetrahydrobenzo[b]thiophen-3-yl)[3-(trifluoromethyl)phenyl]methanone was prepared as described above, starting from cyclohexanone and 3-trifluoromethylbenzoylacetonitrile: mp 122-123° C., ¹H NMR (CDCl₃) δ 1.41-1.63 (m, 2H, CH₂), 1.68-1.79 (m, 4H, 2×CH₂), 2.47-2.54 (m, 2H, CH₂), 7.00 (bs, 2H, NH₂), 7.48-7.72 (m, 4H, Hₐᵣₒₘ).

### LUF5473

(2-Amino-4,5,6,7-tetrahydrobenzo[b]thiophen-3-yl)(3-chlorophenyl)methanone was prepared as described above, starting from cyclohexanone and 3-chlorobenzoylacetonitrile: mp 114-115° C., ¹H NMR (CDCl₃) δ 1.42-1.58 (m, 2H, CH₂), 1.70-1.87 (m, 4H, 2×CH₂), 2.47-2.55 (m, 2H, CH₂), 6.88 (bs, 2H, NH₂), 7.31-7.44 (m, 4H, Hₐᵣₒₘ).

### LUF5533

(2-Amino-4,5,6,7-tetrahydrobenzo[b]thiophen-3-yl)(3-iodophenyl)methanone was prepared as described above, starting from cyclohexanone and 3-iodobenzoylacetonitrile: mp 160-162° C., ¹H NMR (CDCl₃) δ 1.47-1.82 (m, 6H, 3×CH₂), 2.49-2.55 (m, 2H, CH₂), 6.80 (bs, 2H, NH₂), 7.14-7.79 (m, 4H, Hₐᵣₒₘ).

### LUF5489

(2-Amino-4,5,6,7-tetrahydrobenzo[b]thiophen-3-yl)[4-(trifluoromethyl)phenyl]methanone was prepared as described above, starting from cyclohexanone and 4-trifluoromethylbenzoylacetonitrile: mp 145-147° C., ¹H NMR (CDCl₃) δ 1.48-1.50 (m, 2H, CH₂), 1.69-1.76 (m, 4H, 2×CH₂), 2.48 (m, 2H, CH₂), 6.91 (bs, 2H, NH₂), 7.54-7.69 (m, 4H, Hₐᵣₒₘ).

### LUF5480

(2-Amino-4,5,6,7-tetrahydrobenzo[b]thiophen-3-yl)(4-chlorophenyl)methanone was prepared as described above, starting from cyclohexanone and 4-chlorobenzoylacetonitrile: mp 140-142° C., ¹H NMR (CDCl₃) δ 1.47-1.52 (m, 2H, CH₂), 1.71-1.81 (m, 4H, 2×CH₂), 2.47-2.54 (m, 2H, CH₂), 6.71 (bs, 2H, NH₂), 7.34-7.45 (m, 4H, Hₐᵣₒₘ).

### LUF5490

(2-Amino-4,5,6,7-tetrahydrobenzo[b]thiophen-3-yl)(4-bromophenyl)methanone was prepared as described above, starting from cyclohexanone and 4-bromobenzoylacetonitrile: mp 152-153° C., ¹H NMR (CDCl₃) δ 1.46-1.54 (m, 2H, CH₂), 1.67-1.83 (m, 4H, 2×CH₂), 2.46-2.51 (m, 2H, CH₂), 6.80 (bs, 2H, NH₂), 7.33-7.55 (m, 4H, Hₐᵣₒₘ).

### LUF5528

(2-Amino-4,5,6,7-tetrahydrobenzo[b]thiophen-3-yl)(4-iodophenyl)methanone was prepared as described above, starting from cyclohexanone and 4-iodobenzoylacetonitrile: mp 150-152° C., ¹H NMR (CDCl₃) δ 1.41-1.87 (m, 6H, 3×CH₂), 2.43-2.52 (m, 2H, CH₂), 6.82 (bs, 2H, NH₂), 7.17-7.75 (m, 4H, Hₐᵣₒₘ).

### LUF5527

(2-Amino-4,5,6,7-tetrahydrobenzo[b]thiophen-3-yl)(4-methylphenyl)methanone was prepared as described above, starting from cyclohexanone and 4-methylbenzoylacetonitrile: mp 155-156° C., ¹H NMR (CDCl₃) δ 1.45-1.50 (m, 2H, CH₂), 1.70-1.91 (m, 4H, 2×CH₂), 2.38 (s, 3H, CH₃) 2.47-2.54 (m, 2H, CH₂), 6.60 (bs, 2H, NH₂), 7.16-7.42 (m, 4H, Hₐᵣₒₘ).

### LUF5526

(2-Amino-4,5,6,7-tetrahydrobenzo[b]thiophen-3-yl)(4-nitrophenyl)methanone was prepared as described above, starting from cyclohexanone and 4-nitrobenzoylacetonitrile: mp 202-204° C., ¹H NMR (CDCl₃) δ 0.85-1.16 (m, 6H, 3×CH₂), 1.88-2.00 (m, 2H, CH₂), 6.43 (bs, 2H, NH₂), 7.01-7.69 (m, 4H, Hₐᵣₒₘ).

### LUF5492

Methyl 4[(2-Amino-4,5,6,7-tetrahydrobenzo[b]thiophen-3-yl)-carbonyl]benzoate was prepared as described above, starting from cyclohexanone and methyl 4-(2-cyanoacethyl)benzoate: mp 159-160° C., ¹H NMR (CDCl₃) δ 1.54 (s, 3H, CH₃), 2.13 (s, 3H, CH₃), 6.40 (bs, 2H, NH₂), 7.39-7.54 (m, 5H, Hₐᵣₒₘ).

### LUF5493

4-[(2-Amino-4,5,6,7-tetrahydrobenzo[b]thiophen-3-yl)-carbonyl]benzoic acid was prepared as described above, starting from cyclohexanone and methyl 4-(2-cyanoacetyl)benzoate and hydrolisation: mp 232-233° C., ¹H NMR (CD₃OD) δ 1.40-1.49 (m, 2H CH₂), 1.64-1.70 (m, 4H, 2×CH₂), 2.42-2.48 (m, 2H, CH₂), 7.43-8.09 (m, 4H, Hₐᵣₒₘ).

### LUF5484

(2-Amino-4,5,6,7-tetrahydrobenzo[b]thiophen-3-yl)(3,4-dichlorophenyl)methanone was prepared as described above, starting from cyclohexanone and 3,4-dichlorobenzoylacetonitrile: mp 152-154° C., ¹H NMR (CDCl₃) δ 1.46-1.54 (m, 2H, CH₂), 1.67-1.83 (m, 4H, 2×CH₂), 2.45-2.51 (m, 2H, CH₂), 7.11 (bs, 2H, NH₂), 7.27-7.56 (m, 3H, Hₐᵣₒₘ).

### LUF5485

### Ethyl 2-amino-4,5,6,7-tetrahydrobenzo[b]thiophene-3-carboxylate

Mp 114-117° C.

### LUF5488

### (2-Amino-5-phenyl-3-thienyl)(4-chlorophenyl)methanone

mp 173-175° C.

### LUF5496

### 2-({5-Amino-4-[3-(trifluoromethyl)benzoyl]-2-thienyl}methyl)-1,3-isoindolinedione

mp 199-200° C.

### Biology

A primary function of certain cell surface receptors is to activate second messenger systems upon binding of an agonist. The S1P₁ receptor selectively binds to and activates Gᵢ-proteins and activation of this receptor subtype thus results in the inhibition of cAMP production. The S1P₂ receptor predominantly activates G_{q}-proteins finally resulting in increases in intracellular calcium. S1P₃ receptor activation results in both, an inhibition of cAMP production and an increase in intracellular calcium, via the activation of Gᵢ- and G_{q}-proteins

Accordingly, we performed measurements on cAMP accumulation (Lance cAMP 384 kit, Perkin Elmer, Zaventem, Belgium) and measurements on changes in intracellular calcium (for method see Jongsma et al., 2006) (8).

TABLE 1

| | | efficacy | | efficacy |
|---|---|---|---|---|
| compound | pEC₅₀ S1P₃ | (α) S1P₃ | pEC₅₀ S1P₁ | (α) S1P₁ |
| LUF5465 | <5 (n = 3) | >0.5 | N.D. (n = 2) | N.D. (n = 2) |
| | | (n = 3) | | |
| LUF5469 | 5.89 +/− | 1.07 +/− | N.D. (n = 2) | N.D. (n = 2) |
| | 0.12 (n = 2) | 0.03 (n = 2) | | |
| LUF5473 | 5.73 +/− | 1.07 +/− | 6.00 +/− | 0.40 +/− |
| | 0.08 | 0.03 (n = 11) | 0.09 (n = 9) | 0.04 (n = 9) |
| | (n = 11) | | | |
| LUF5480 | 5.17 +/− | 0.37 +/− | N.D. (n = 4) | N.D. (n = 4) |
| | 0.22 (n = 5) | 0.11 (n = 5) | | |
| LUF5489 | <5 (n = 1) | N.D. (n = 1) | N.D. (n = 1) | N.D. (n = 1) |
| LUF5490 | 5.94 +/− | 0.41 +/− | N.D. (n = 3) | N.D. (n = 3) |
| | 0.20 (n = 3) | 0.05 (n = 3) | | |
| LUF5492 | 6.03 +/− | 0.25 +/− | N.D. (n = 3) | N.D. (n = 3) |
| | 0.45 (n = 2) | 0.10 (n = 2) | | |
| LUF5527 | 5.51 +/− | 0.96 +/− | N.D. (n = 3) | N.D. (n = 3) |
| | 0.13 (n = 3) | 0.09 (n = 3) | | |
| LUF5528 | <5 (n = 2) | <0.5 (n = 2) | N.D. (n = 2) | N.D. (n = 2) |
| LUF5532 | 5.38 +/− | 0.71 +/− | 5.38 +/− | 0.63 +/− |
| | 0.02 (n = 3) | 0.11 (n = 3) | 0.47 (n = 3) | 0.26 (n = 3) |
| LUF5533 | 5.90 +/− | 0.94 +/− | 5.86 +/− | 0.46 +/− |
| | 0.03 (n = 2) | 0.00 (n = 2) | 0.32 (n = 2) | 0.04 (n = 2) |
| LUF5464 | <5 (n = 3) | +/−1 (n = 3) | N.D. (n = 3) | N.D. (n = 3) |
| LUF5484 | <5 (n = 2) | <0.5 (n = 2) | N.D. (n = 2) | N.D. (n = 2) |
| LUF5485 | N.D. (n = 3) | N.D. (n = 3) | N.D. (n = 3) | N.D. (n = 3) |
| LUF5488 | N.D. (n = 2) | N.D. (n = 2) | N.D. (n = 2) | N.D. (n = 2) |
| LUF5496 | <5 (n = 2) | N.D. (n = 2) | N.D. (n.2) | N.D. (n = 2) |
| S1P | 9.46 +/− | 1.00 +/− | 9.86 +/− | 1.00 +/− |
| | 0.11 | 0.03 (n = 11) | 0.10 (n = 10) | 0.05 (n = 10) |
| | (n = 11) | | | |

N.D. Not defined because the potency and/or the efficacy were too low

### Structure Activity Relationships

In Table 1 results of the cAMP accumulation assay at the S1P₁ and S1P₃ receptor are displayed for a number of compounds, in comparison with results for S1P itself. Compounds with efficacy at the S1P₂ receptor also induced S1P₂-mediated changes in intracellular calcium but generally with lower potency (data not shown). None of the compounds tested showed efficacy at the S1P₂ receptor.

### LIST OF REFERENCES

(1) Chun J, Goetzl E J, Hla T, Igarashi Y, Lynch K R, Moolenaar W, Pyne S, Tigyi G (2002) International Union of Pharmacology. XXXIV. Lysophospholipid receptor nomenclature. Pharmacol Rev, 54, 265-269.
(2) Sanchez T and Hla T (2004) Structural and functional characteristics of S1P receptors. J Cell Biochem, 92, 913-922.
(3) Forrest M, Sun S Y, Hajdu R, Bergstrom J, Card D, Doherty G, Hale J, Keohane C, Meyers C, Milligan J, Mills S, Nomura N, Rosen H, Rosenbach M, Shei G J, Singer I I, Tian M, West S, White V, Xie J, Proia R L, Mandala S (2004) Immune cell regulation and cardiovascular effects of sphingosine 1-phosphate receptor agonists in rodents are mediated via distinct receptor subtypes. J Pharmacol Exp Ther, 309, 758-768.
(4) Sanna M G, Liao J, Jo E, Alfonso C, Ahn M Y, Peterson M S, Webb B, Lefebvre S, Chun J, Gray N, Rosen H (2004) Sphingosine 1-phosphate (S1P) receptor subtypes S1P1 and S1P3, respectively, regulate lymphocyte recirculation and heart rate. J Biol Chem, 279, 13839-13848.
(5) Theilmeier G, Schmidt C, Herrmann J, Keul P, Schafers M, Herrgott I, Mersmann J, Larmann J, Hermann S, Stypmann J, Schober O, Hildebrand R, Schulz R, Heusch G, Haude M, von Wnuck Lipinski K, Herzog C, Schmitz M, Erbel R, Chun J, Levkau B (2006) High-density lipoproteins and their constituent, sphingosine-1-phosphate, directly protect the heart against ischemia/reperfusion injury in vivo via the S1P3 lysophospholipid receptor. Circulation. 114:1403-9.
(6) Keul P, Tolle M, Lucke S, von Wnuck Lipinski K, Heusch G, Schuchardt M, van der Giet M, Levkau B (2007) The Sphingosine-1-Phosphate Analogue FTY720 Reduces Atherosclerosis in Apolipoprotein E-Deficient Mice. Arterioscler Thromb Vasc Biol. (Epub)
(7) Van der Klein, P A M, Kourounakis, A P, IJzerman, A P (1999) Allosteric modulation of the A1 receptor. Synthesis and biological evaluation of novel 2-amino-3-benzoylthiophenes as allosteric enhancers of agonist binding. J Med Chem, 42, 3629-3635.
(8) Jongsma M, Hendriks-Balk M C, Michel M C, Peters S L, Alewijnse A E (2006) BML-241 fails to display selective antagonism at the sphingosine-1-phosphate receptor, S1P (3). Br J Pharmacol, 149, 277-282.
(9) Linden, Joel M.; Ollson, Ray A.; Scammells, Peter J. Preparation 2-amino-3-aroyl-4,5-alkylthiophenes which are agonist allosteric enhancers at human A1 adenosine receptors. U.S. Pat. No. 6,713,638.
(10) Nikolakopoulos, George; Figler, Heidi; Linden, Joel; Scammells, Peter J. 2-Aminothiophene-3-carboxylates and carboxamides as adenosine A1 receptor allosteric enhancers. Bioorganic & Medicinal Chemistry (2006), 14(7), 2358-2365.
(11) Baraldi, Pier Giovanni; Pavani, Maria Giovanna; Leung, Edward; Moorman, Allan R.; Varani, Katia; Vincenzi, Fabrizio; Borea, Pier Andrea; Romagnoli, Romeo. Synthesis and biological characterization of [3H] (2-amino-4,5,6,7-tetrahydrobenzo[b]thiophen-3-yl)(4-chlorophenyl)methanone, the first radiolabeled adenosine A1 allosteric enhancer. Bioorganic & Medicinal Chemistry Letters (2006), 16(5), 1402-1404.
(12) Baraldi, Pier Giovanni; Pavani, Maria Giovanna; Shryock, John C.; Moorman, Allan R.; Iannotta, Valeria; Borea, Pier Andrea; Romagnoli, Romeo. Synthesis of 2-amino-3-heteroaroylthiophenes and evaluation of their activity as potential allosteric enhancers at the human A1 receptor. European Journal of Medicinal Chemistry (2004), 39(10), 855-865.
(13) Baraldi, Pier Giovanni. Preparation of thienylmethanones as allosteric adenosine receptor modulators. U.S. Pat. No. 6,727,258
(14) Bruns, Robert F.; Fergus, James H.; Coughenour, Linda L.; Courtland, Geneva G.; Pugsley, Thomas A.; Dodd, John H.; Tinney, Francis J. Structure-activity relationships for enhancement of adenosine A1 receptor binding by 2-amino-3-benzoylthiophenes. Molecular Pharmacology (1990), 38(6), 950-8.

1.-18. (canceled)

19. A compound of the general formula (1): 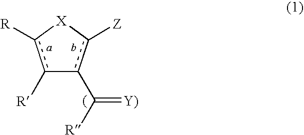
(1)
or a salt thereof,
wherein
X represents oxygen sulphur, NH or C1-C4 alkyl substituted NH;
C═Y is optional but when present, Y represents oxygen, sulphur, NH, or C1-C4 alkyl substituted NH:
Z represents hydrogen, halogen, amide, hydroxyl, substituted or unsubstituted alkyl, substituted or unsubstituted alkenyl, substituted or unsubstituted aryl or heteroaryl, substituted or unsubstituted aralkyl, alkoxyl, amino, mono- or di-C1-C4 alkyl substituted amino, sulphydryl, carbonyl, carboxyl, acrylate, methacrylate, vinyl, styryl, sulphonate, sulphonic acid, or quaternary ammonium;
R represents hydrogen, halogen, substituted or unsubstituted alkyl, substituted or unsubstituted alkenyl, substituted or unsubstituted alkynyl, or substituted or unsubstituted —(CH2)n-aryl;
R′ represents hydrogen, halogen, substituted or unsubstituted alkyl, substituted or unsubstituted alkenyl, substituted or unsubstituted alkynyl, or substituted or unsubstituted —(CH2)n-aryl; or R,R′ are linked together to form a ring which may be saturated or unsaturated, substituted or unsubstituted and has three to seven members;
R″ represents hydrogen, —(CH2)n-hydroxyl, halogen, acyl, thio-acyl, seleno-acyl, (substituted) alkyl, (substituted) alkenyl, (substituted) alkynyl, or (substituted) —(CH2)n-aryl;
bonds a and b, shown in dashed line, may be present or absent;
and n is a number in the range of from 0 to 10.

20. The compound or a salt thereof according to claim 19 wherein Z is substituted or unsubstituted alkyl, alkoxy, aralkyl or alkenyl and the alkyl or alkenyl group of Z is C1-C4.

21. The compound or a salt thereof according to claim 19 wherein Z is NH2.

22. The compound or a salt thereof according to claim 19 wherein X is sulphur.

23. The compound or a salt thereof according to claim 19 wherein Y is oxygen.

24. The compound or a salt thereof according to claim 19 wherein the compound of formula (1) has the structure of formula (2): 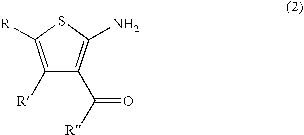
(2)
wherein R, R′ and R″ have the same meaning as in claim 1.

25. The compound or a salt thereof according to claim 19 wherein the compound of formula (1) has the structure of formula (3): 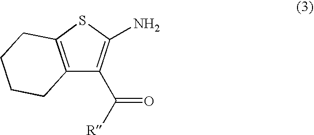
(3)
and wherein R″ has the same meaning as in claim 19.

26. The compound or a salt thereof according to claim 25 wherein R″ is phenyl or substituted phenyl.

27. The compound or a salt thereof according to claim 25 wherein the compound of formula (3) is selected from the group consisting of:
(2-Amino-4,5,6,7-tetrahydrobenzo[b]thiophen-3-yl)(4-chlorophenyl)methanone;
(2-Amino-4,5,6,7-tetrahydrobenzo[b]thiophen-3-yl)(4-bromophenyl)methanone;
Methyl 4[(2-Amino-4,5,6,7-tetrahydrobenzo[b]thiophen-3-yl)-carbonyl]benzoate;
(2-Amino-4,5,6,7-tetrahydrobenzo[b]thiophen-3-yl)(2-chlorophenyl)methanone;
(2-Amino-4,5,6,7-tetrahydrobenzo[b]thiophen-3-yl)(3-chlorophenyl)methanone;
(2-Amino-4,5,6,7-tetrahydrobenzo[b]thiophen-3-yl)(3-iodophenyl)methanone;
(2-Amino-4,5,6,7-tetrahydrobenzo[b]thiophen-3-yl)(4-methylphenyl)methanone;
(2-Amino-4,5,6,7-tetrahydrobenzo[b]thiophen-3-yl)[3-(trifluoromethyl)phenyl]methanone; and salts thereof.

28. A pharmaceutical composition comprising as active ingredient one or more compounds or salts thereof as defined in claim 19.

29. A method of treatment of an S1P receptor mediated condition comprising the administration of an effective amount of a compound or a salt thereof, as defined in claim 19, to a subject.

30. A method of treatment of an S1P receptor mediated condition comprising the administration of an effective amount of a compound or a salt thereof, as defined in claim 20, to a subject.

31. A method of treatment of an S1P receptor mediated condition comprising the administration of an effective amount of a compound or a salt thereof, as defined in claim 21, to a subject.

32. A method of treatment of an S1P receptor mediated condition comprising the administration of an effective amount of a compound or a salt thereof, as defined in claim 22, to a subject.

33. A method of treatment of an S1P receptor mediated condition comprising the administration of an effective amount of a compound or a salt thereof, as defined in claim 23, to a subject.

34. A method of treatment of an S1P receptor mediated condition comprising the administration of an effective amount of a compound or a salt thereof, as defined in claim 24, to a subject.

35. A method of treatment of an S1P receptor mediated condition comprising the administration of an effective amount of a compound or a salt thereof, as defined in any one of claim 25, to a subject.

36. A method of treatment of an S1P receptor mediated condition comprising the administration of an effective amount of a compound or a salt thereof, as defined in claim 26, to a subject.

37. A method of treatment of an S1P receptor mediated condition comprising the administration of an effective amount of a compound or a salt thereof, as defined in claim 27, to a subject.

38. A method of treatment according to claim 29 wherein the condition is an S1P3 mediated condition.

39. A method of treatment according to claim 38 wherein the condition is selected from the group consisting of cardiovascular diseases, atherosclerosis, cancer, pulmonary oedema, autoimmune disorders and Adult Respiratory Distress Syndrome.
